(19) United States
(12) Patent Application Publication
(10) Pub. No.: US 2026/0306187 A1
(43) Pub. Date: Oct. 8, 2026

(54) SYSTEM AND METHOD FOR UPDATING REGISTRATION AND LOCALIZATION DURING SURGICAL NAVIGATION

(71) Applicant: Covidien LP, Mansfield, MA (US)

(72) Inventors: Dany Junio, Brookline, MA (US); Jing Zhao, Superior, CO (US)

(21) Appl. No.: 19/485,572

(22) PCT Filed: May 21, 2024
(86) PCT No.: PCT/IB2024/054932

§ 371 Date: Nov. 18, 2025

Related U.S. Application Data
(60) Provisional application No. 63/468,578, filed on May 24, 2023.

(51) Int. Cl. A61B 34/20

(52) U.S. Cl. CPC A61B 34/20; A61B 2034/2051

(57) ABSTRACT

A surgical navigation system includes a navigation catheter and a computing device. The navigation catheter includes a force sensor. The computing device is configured to register location data of the navigation catheter to image data of the luminal network, receive force measurements from the force sensor, compare the force measurements to expected force measurements and determine whether the force measurements deviate from expected force measurements along the path, and update registration of the location data to the image data if it is determined that a force measurement deviates from an expected force measurement along the path.

### CROSS-REFERENCE TO RELATED APPLICATION

This application claims the benefit of U.S. Provisional Patent Application Ser. No. 63/468,578, filed May 24, 2023, the entire content of which is incorporated herein by reference.

### FIELD

This disclosure relates to systems, methods, and devices for dynamically updating registration between location data and image data, and dynamically updating image data, during a surgical navigation procedure based on force measurements.

### BACKGROUND

There are several commonly applied medical methods, such as endoscopic procedures or minimally invasive procedures, for treating various maladies affecting organs including the liver, brain, heart, lungs, gall bladder, kidneys, and bones. Often, one or more imaging modalities, such as magnetic resonance imaging (MRI), ultrasound imaging, computed tomography (CT), or fluoroscopy are employed by clinicians to identify and navigate to areas of interest within a patient and ultimately a target for biopsy or treatment. In some procedures, pre-operative scans may be utilized for target identification and intraoperative guidance.

For example, an endoscopic approach has proven useful in navigating to areas of interest within a patient, and particularly so for areas within luminal networks of the body such as the lungs. To enable the endoscopic approach, and more particularly the bronchoscopic approach in the lungs, endobronchial navigation systems have been developed that use previously acquired MRI data or CT image data to generate a three-dimensional (3D) rendering, model, or volume of the particular body part such as the lungs. The resulting volume generated from the MRI scan or CT scan may be utilized to create a navigation plan to facilitate the advancement of a navigation catheter (or other suitable medical device) through a bronchoscope and a branch of the bronchus of a patient to an area of interest. A locating or tracking system, such as an electromagnetic (EM) tracking system, may be utilized in conjunction with, for example, image data, to facilitate guidance of the navigation catheter through the branch of the bronchus to the area of interest. In certain instances, the navigation catheter may be positioned within one of the airways of the branched luminal networks adjacent to, or within, the area of interest to provide access for one or more medical instruments.

However, when navigating inside the lungs per a pre-operative, image-based plan, the shape of the lungs is not precisely as it was when the pre-operative image data was acquired. This mis-match between the image data and the shape of the lungs during the navigation procedure is known as CT to body divergence. Existing solutions for addressing CT to body divergence require capturing new images of the patient during the navigation procedure to realign the image data with the patient's lungs, which is time consuming and exposes the patient to additional and unnecessary radiation.

### SUMMARY

Systems and methods for localization and divergence correction in a surgical navigation procedure using a force sensor are provided.

According to an aspect of this disclosure, a surgical navigation system includes a surgical navigation catheter and a computing device. The surgical navigation catheter includes a force sensor located at a distal portion of the surgical navigation catheter. The computing device includes a processor and memory storing instructions which, when executed by the processor, cause the computing device to: register location data, corresponding to locations of the surgical navigation catheter navigating along a path through a luminal network, to image data of the luminal network; receive force measurements from the force sensor of the surgical navigation catheter; compare force measurements received from the force sensor to expected force measurements along the path and determine whether force measurements received from the force sensor deviate from expected force measurements along the path; and update registration of the location data to the image data of the luminal network if it is determined that a force measurement received from the force sensor deviates from an expected force measurement along the path.

In an aspect, the computing device may be further configured to acquire force measurements of an area around a distal portion of the surgical navigation catheter if it is determined that a force measurement received from the force sensor deviates from an expected force measurement along the path, and generate a point cloud of the area around the distal portion of the surgical navigation catheter based on the acquired force measurements.

In an aspect, the computing device may be further configured to determine an area of the image data that corresponds to the generated point cloud, and update registration of the location data to the image data of the luminal network based on the determined area.

In an aspect, the computing device may be further configured to determine an area of the image data that corresponds to the generated point cloud, calculate an offset between the determined area and the generated point cloud, and update registration of the location data to the image data of the luminal network based on the calculated offset.

In an aspect, the computing device may be further configured to update at least a portion of the image data based on the generated point cloud.

In an aspect, the computing device may be further configured to determine an area of the image data that corresponds to the generated point cloud, calculate a distance between an edge of the area of the image data and an edge of the generated point cloud, and update registration of the location data to the image data of the luminal network based on the calculated distance.

In an aspect, the system may further include a display operably coupled to the computing device. The computing device may be configured to cause the display to display a rendering of the luminal network based on the image data, and a rendering of the surgical navigation catheter positioned relative to the rendering of the luminal network based on the updated registration of the location data to the image data.

In an aspect, the computing device may be further configured to generate a notification if it is determined that a force measurement received from the force sensor deviates from an expected force measurement along the path.

In an aspect, the computing device may be configured to initiate movement of the surgical navigation catheter if it is determined that a force measurement received from the force sensor deviates from an expected force measurement along the path.

In accordance with another aspect of the disclosure, a surgical navigation system includes a surgical navigation catheter, a tracking system operably coupled to the surgical navigation catheter, and a computing device operably coupled to the surgical navigation catheter and the tracking system. The surgical navigation catheter includes a location sensor and a force sensor and is configured to navigate along a path through a luminal network. The tracking system is operably coupled to the surgical navigation catheter and is configured to generate location data corresponding to locations of the surgical navigation catheter within the luminal network based on signals received from the location sensor as the surgical navigation catheter is navigated through the luminal network. The computing device includes a processor and memory storing instructions which, when executed by the processor, cause the computing device to register the generated location data to image data of the luminal network, compare force measurements received from the force sensor to expected force measurements along the path and determine whether force measurements received from the force sensor deviate from expected force measurements along the path, and update registration of the generated location data to the image data of the luminal network if it is determined that a force measurement received from the force sensor deviates from an expected force measurement along the path.

In an aspect, the location sensor and the force sensor may be disposed at a distal portion of the surgical navigation catheter.

In an aspect, the computing device may be configured to acquire force measurements of an area around a distal portion of the surgical navigation catheter if it is determined that a force measurement received from the force sensor deviates from an expected force measurement along the path, and generate a point cloud of the area around the distal portion of the surgical navigation catheter based on the acquired force measurements.

In an aspect, the computing device may be configured to determine an area of the image data that corresponds to the generated point cloud, and update registration of the generated location data to the image data of the luminal network based on the determined area.

In an aspect, the computing device may be configured to determine an area of the image data that corresponds to the generated point cloud, calculate an offset between the determined area and the generated point cloud, and update registration of the generated location data to the image data of the luminal network based on the calculated offset.

In an aspect, the computing device may be configured to update at least a portion of the image data based on the generated point cloud.

In an aspect, the system may further include a display operably coupled to the computing device. The computing device may be configured to cause the display to display a rendering of the luminal network based on the image data, and a rendering of the surgical navigation catheter positioned relative to the rendering of the luminal network based on the updated registration of the generated location data to the image data.

In an aspect, the computing device may be configured to generate a notification if it is determined that a force measurement received from the force sensor deviates from an expected force measurement along the path.

In an aspect, the computing device may be configured to initiate movement of the surgical navigation catheter if it is determined that a force measurement received from the force sensor deviates from an expected force measurement along the path.

In accordance with another aspect of the disclosure, a method for enhanced navigation of a surgical navigation catheter along a path of a luminal network is provided. The method includes comparing force measurements received from a force sensor of the surgical navigation catheter to expected force measurements along the path of the luminal network and determining whether force measurements received from the force sensor deviate from expected force measurements along the path, acquiring force measurements of an area around a distal portion of the surgical navigation catheter if it is determined that a force measurement received from the force sensor deviates from an expected force measurement along the path, generating a point cloud of the area around the distal portion of the surgical navigation catheter based on the acquired force measurements, and updating registration of the location data, corresponding to locations of the surgical navigation catheter navigating along the path, to image data of the luminal network based on the generated point cloud.

In an aspect, the method may further include displaying a rendering of the luminal network based on the image data, and displaying a rendering of the surgical navigation catheter positioned relative to the displayed rendering of the luminal network based on the updated registration of the location data to the image data.

Any of the above aspects and embodiments of this disclosure may be combined without departing from the scope of this disclosure.

### BRIEF DESCRIPTION OF THE DRAWINGS

Objects and features of the system and method disclosed herein will become apparent to those of ordinary skill in the art when descriptions of various embodiments thereof are read with reference to the accompanying drawings, of which:

FIG. 1 is a schematic diagram of a surgical navigation procedure system in accordance with an illustrative aspect of this disclosure;

FIG. 2 is a schematic diagram of a computing device which forms part of the surgical navigation procedure system of FIG. 1 in accordance with an aspect of this disclosure;

FIG. 3 is a view of a distal portion of a flexible surgical navigation catheter having a force sensor in accordance with an aspect of this disclosure;

FIG. 4 is a flowchart illustrating a method for dynamic localization and registration in accordance with an aspect of this disclosure;

FIG. 5A illustrates comparative views of location data of a tracked surgical navigation catheter relative to a three-dimensional model of image data before dynamic updating of registration between the location data and the image data is applied in accordance with an aspect of this disclosure; and

FIG. 5B illustrates comparative views of location data of the tracked surgical navigation catheter relative to the three-dimensional model of image data after dynamic updating of registration between the location data and the image data is applied in accordance with an aspect of this disclosure.

### DETAILED DESCRIPTION

Although this disclosure will be described in terms of specific illustrative embodiments, it will be readily apparent to those skilled in the art that various modifications, rearrangements, and substitutions may be made without departing from the spirit of this disclosure. The scope of this disclosure is defined by the claims appended hereto.

This disclosure provides a system and method for dynamically updating registration between location data, corresponding to a location of a surgical navigation catheter, and image data during a surgical navigation procedure. Updating registration is based on force measurements acquired by a force sensor located at a distal tip of the surgical navigation catheter. In particular, this disclosure utilizes force measurements acquired by a force sensor located at the distal tip of a surgical navigation catheter as the catheter is being navigated along a planned pathway within a patient's lungs. The acquired force data serves to detect the position of the distal tip of the catheter relative to a planned pathway and three-dimensional data set. For example, the force sensor at the distal tip may sense a force applied as the tip of the catheter abuts the wall of the lumen in which the catheter is positioned when such a force is not expected or intended. Alternatively, the system can be expecting a force to be imparted against the distal tip of the catheter based on an assumed location of the catheter and an expectation that the catheter should be abutting a wall in the assumed location, but the force sensor at the distal tip senses no force being imparted upon the catheter. In either scenario, the system actuates movement of the distal tip of the catheter to map the lumen in which it is positioned based on force measurements acquired during the actuation to generate a point cloud, then compares the generated point cloud to the plan and image data to alert of changes and/or to update the pre-planned map based on the newly detected changes.

The force data acquired by the force sensor of the catheter enables radiation-free registration updating, may be utilized to generate alerts and notifications, and may be utilized to improve navigation of surgical devices within a patient's luminal network. Additionally, the force sensor may be used for tissue-type recognition (e.g., malignant vs. benign) based on the sensing of the tissue's mechanical properties.

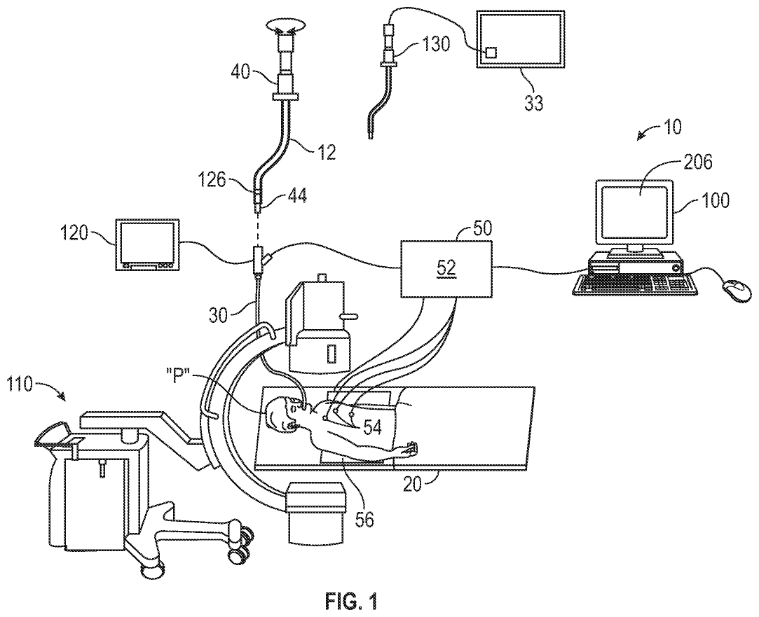

FIG. 1 depicts a surgical navigation system 10 configured for reviewing image data (e.g., CT image data) to identify one or more targets, planning a pathway to an identified target (planning phase), navigating a catheter 12 (e.g., an extended working channel) of a catheter guide assembly 40 to a target (navigation phase) via a user interface, and confirming placement of the catheter 12 relative to the target. One such system may be an Electromagnetic Navigation (EMN) system such as the ELECTROMAGNETIC NAVIGATION BRONCHOSCOPY® navigation system currently sold by Medtronic plc. The target may be tissue of interest identified by review of the CT image data during the planning phase. Following navigation, a medical instrument, such as a biopsy tool, ablation tool (e.g., ablation device 130), or other tool, may be inserted into the catheter 12 to treat the tissue or obtain a tissue sample from the tissue located at, or proximate to, the target. Although CT image data is described, any form of imaging data by any imaging device may be utilized prior to, or during, a procedure.

System 10 generally includes an operating table 20 configured to support the patient “P;” a bronchoscope 30 configured for insertion through patient's “P's” mouth into patient's “P's” airways; monitoring equipment including a display 120 coupled to bronchoscope 30 (e.g., a video display, for displaying the video images received from the video imaging system of bronchoscope 30); a tracking system 50 including a tracking module 52, a plurality of reference sensors 54 and a transmitter mat 56; and a computing device 100 including software and/or hardware used to facilitate identification of a target, pathway planning to the target, navigation of a medical instrument to the target, and confirmation of placement of the catheter 12, or a suitable device therethrough, relative to the target.

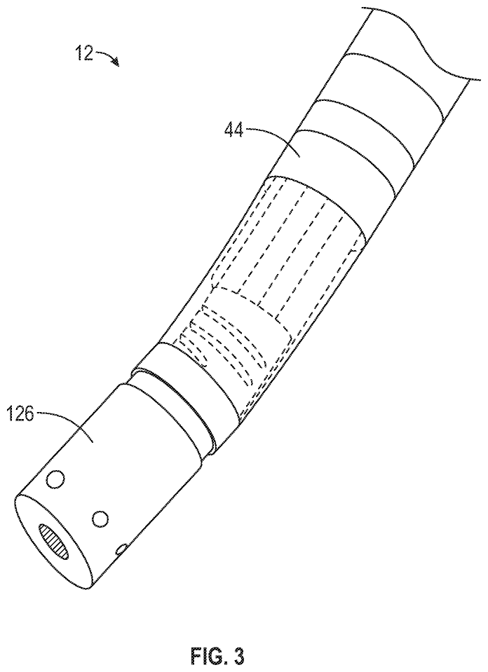

As shown in FIG. 1 and with brief reference to FIG. 3, catheter 12 is part of a catheter guide assembly 40. In practice, catheter 12 is inserted into bronchoscope 30 for access to a luminal network of patient “P.” Specifically, catheter 12 of catheter guide assembly 40 may be inserted into a working channel of bronchoscope 30 for navigation through a patient's luminal network. A distal portion of the catheter 12 includes a location sensor 44 and a force sensor 126. The position and orientation of the location sensor 44 within an electromagnetic field, and thus, the distal portion of the catheter 12 relative to a reference coordinate system, can be derived by the tracking system 50. The force sensor 126 may be any type of sensing device capable of sensing and/or measuring the bending and torsional forces exerted upon the distal tip of the catheter, such as semiconductor or metallic foil strain gauges. As described in greater detail below, computing device 100 utilizes the force sensor 126 measurements to determine whether (and the extent to which) the distal tip of the catheter 12 is abutting a wall of a lumen through which the catheter 12 is being navigated or whether the distal tip of the catheter 12 is positioned within the center of the lumen. The computing device 100 also utilizes the force sensor 126 data to generate a point cloud, update registration between data sets, and/or update data sets based on the generated point cloud.

An imaging device 110 capable of acquiring images or video of patient “P” (e.g., fluoroscopic, x-ray, MRI, CT, ultrasonic, etc.) may also be included in this particular aspect of system 10. The image data (e.g., images, series of images, or video) captured by the imaging device 110 may be stored within the imaging device 110 or transmitted to computing device 100 for storage, processing, and display. Additionally, the imaging device 110 may move relative to patient “P” so that images may be acquired from different angles or perspectives relative to patient “P” to create a video from a sweep.

Computing device 100 may be any suitable computing device including a processor and storage medium, wherein the processor is capable of executing instructions stored on the storage medium. The computing device 100 may further include a database configured to store patient data, image data sets including CT images, fluoroscopic data sets including fluoroscopic images and video, navigation plans, and any other such data. Although not explicitly illustrated, the computing device 100 may include inputs, or may otherwise be configured to receive, image data sets, fluoroscopic images/video and other data described herein. Additionally, computing device 100 includes a display (e.g., display 206) configured to display graphical user interfaces.

With respect to the planning phase, computing device 100 utilizes previously acquired image data (e.g., CT image data, MRI image data, etc.) for generating and viewing a three-dimensional model or rendering of patient “P's” airways, enables the identification of a target on the three-dimensional model (automatically, semi-automatically, or manually), and allows for determining a pathway through patient “P's” airways to tissue located at and around the target. More specifically, in an aspect, CT images acquired from previous CT scans are processed and assembled into a three-dimensional CT volume, which is then utilized to generate a three-dimensional model of patient “P's” airways. The three-dimensional model may be displayed on a display 206 associated with computing device 100, or in any other suitable fashion. Using computing device 100, various views of the three-dimensional model or enhanced two-dimensional images generated from the three-dimensional model are presented. The enhanced two-dimensional images may possess some three-dimensional capabilities because they are generated from three-dimensional data. The three-dimensional model may be manipulated to facilitate identification of target on the three-dimensional model or two-dimensional images, and selection of a suitable pathway through patient “P's” airways to access tissue located at the target can be made. Once selected, the pathway plan, three-dimensional model, and images derived therefrom, can be saved and exported to a navigation system for use during the navigation phase(s). One such planning software is the ILLUMISITE® planning suite currently sold by Medtronic plc.

With respect to the navigation phase, the tracking system 50 is utilized for performing registration of the images and the pathway for navigation, although other configurations are also contemplated. As noted above, tracking system 50 includes a tracking module 52, a plurality of reference sensors 54, and a transmitter mat 56 (including markers if applicable). Tracking system 50 is configured for use with a location sensor 44 of catheter 12 and may be configured to track, for example, the electromagnetic position thereof within an electromagnetic coordinate system.

Transmitter mat 56 is positioned beneath patient “P.” Transmitter mat 56 generates an electromagnetic field around at least a portion of patient “P” within which the position of a plurality of reference sensors 54 and the location sensor 44 can be determined with use of a tracking module 52. One or more of reference sensors 54 are attached to the chest of patient “P.” The six degrees of freedom coordinates of reference sensors 54 are sent to computing device 100 (which includes the appropriate software) where they are used to calculate a patient coordinate frame of reference. Registration, as detailed below, is generally performed to coordinate locations of the three dimensional model and two dimensional images from the planning phase with patient's “P's” airways as observed through the bronchoscope 30, and to allow for the navigation phase to be undertaken with precise knowledge of the location of the location sensor 44, even in portions of the airway where the bronchoscope 30 cannot reach.

Registration of patient's “P's” location on the transmitter mat 56 is performed by moving location sensor 44 through the airways of patient's “P.” More specifically, data pertaining to locations of location sensor 44, while catheter 12 is moving through the airways, is recorded using transmitter mat 56, reference sensors 54, and tracking module 52. A shape resulting from this location data is compared to an interior geometry of passages of the three dimensional model generated in the planning phase, and a location correlation between the shape and the three dimensional model based on the comparison is determined, e.g., utilizing the software on computing device 100. In addition, the software identifies non-tissue space (e.g., air filled cavities) in the three dimensional model. The software aligns, or registers, an image representing a location of location sensor 44 with the three dimensional model and two dimensional images generated from the three dimension model, which are based on the recorded location data and an assumption that location sensor 44 remains located in non-tissue space in patient's “P's” airways. Alternatively, a manual registration technique may be employed by navigating the bronchoscope 30 with the location sensor 44 to pre-specified locations in the lungs of patient “P”, and manually correlating the images from the bronchoscope to the model data of the three dimensional model.

Though described herein with respect to EMN systems using EM sensors, the instant disclosure is not so limited and may be used in conjunction with flexible sensor, ultrasonic sensors, or without sensors. Additionally, the methods described herein may be used in conjunction with robotic systems such that robotic actuators drive the catheter 12, catheter guide assembly 40 components, or bronchoscope 30 proximate the target.

Following registration of patient “P” to the image data and pathway plan, a user interface is displayed in the navigation software which sets forth the pathway that the clinician is to follow to reach the target.

Once catheter 12 has been successfully navigated proximate the target as depicted on the user interface, the catheter 12 is in place as a guide channel for guiding medical instruments including without limitation, optical systems, ultrasound probes, marker placement tools, biopsy tools, ablation tools (i.e., microwave ablation devices), laser probes, cryogenic probes, sensor probes, and aspirating needles to the target. In an aspect, ablation device 130 is a flexible surgical navigation catheter which is guided through catheter 12 for placement relative to a target and ablation of the target. Ablation device 130 is configured to connect to microwave generator 33 (FIG. 1) which generates and controls the application of microwave energy through the ablation device 130. Microwave generator 33 may be a component of computing device 100 or may be a separate stand-alone component.

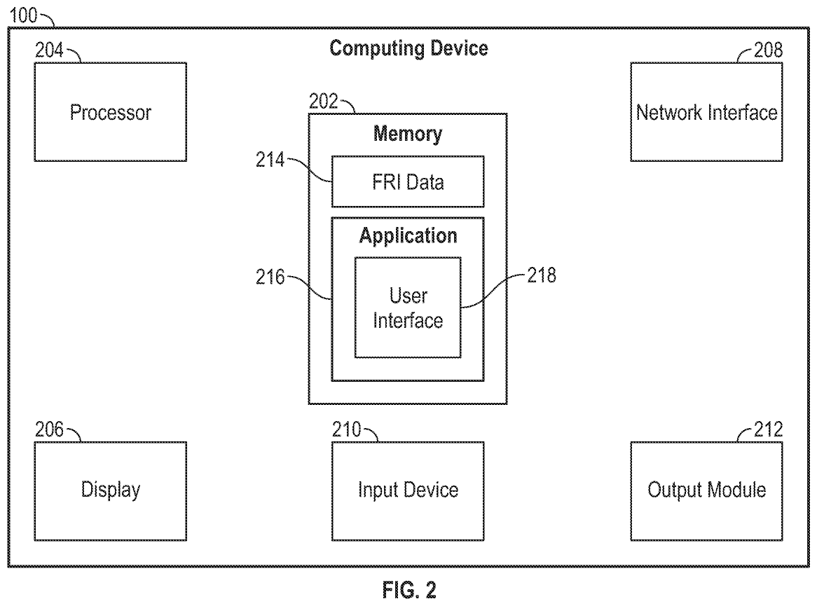

FIG. 2 illustrates a system diagram of computing device 100. Computing device 100 may include memory 202, processor 204, display 206, network interface 208, input device 210, and/or output module 212. Memory 202 includes any non-transitory computer-readable storage media for storing data and/or software that is executable by processor 204 and which controls the operation of computing device 100. In an embodiment, memory 202 may include one or more solid-state storage devices such as flash memory chips. Alternatively or in addition to the one or more solid-state storage devices, memory 202 may include one or more mass storage devices connected to the processor 204 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 204. That is, computer readable storage media includes non-transitory, volatile and non-volatile, removable and non-removable media implemented in any method or technology for storage of information such as computer-readable instructions, data structures, program modules, or other data. For example, computer-readable storage media includes RAM, ROM, EPROM, EEPROM, flash memory or other solid state memory technology, CD-ROM, DVD, Blu-Ray or other optical storage, magnetic cassettes, magnetic tape, magnetic disk storage or other magnetic storage devices, or any other medium which can be used to store the desired information and which can be accessed by computing device 100.

Memory 202 may store application 216 and/or functional respiratory imaging data 214 of one or more patients. Application 216 may, when executed by processor 204, cause display 206 to present user interfaces. Processor 204 may be a general-purpose processor, a specialized graphics processing unit (GPU) configured to perform specific graphics processing tasks while freeing up the general-purpose processor to perform other tasks, and/or any number or combination of such processors. Display 206 may be touch sensitive and/or voice activated, enabling display 206 to serve as both an input and output device. Alternatively, a keyboard (not shown), mouse (not shown), or other data input devices may be employed. Network interface 208 may be configured to connect to a network such as a local area network (LAN) consisting of a wired network and/or a wireless network, a wide area network (WAN), a wireless mobile network, a Bluetooth network, and/or the internet. For example, computing device 100 may receive functional respiratory imaging data, DICOM imaging data, computed tomographic (CT) image data, or other imaging data, of a patient from an imaging workstation and/or a server, for example, a hospital server, internet server, or other similar servers, for use during surgical ablation planning. Patient functional respiratory imaging data may also be provided to computing device 100 via a removable memory 202. Computing device 100 may receive updates to its software, for example, application 216, via network interface 208. Computing device 100 may also display notifications on display 206 that a software update is available.

Input device 210 may be any device by means of which a user may interact with computing device 100, such as, for example, a mouse, keyboard, foot pedal, touch screen, and/or voice interface. Output module 212 may include any connectivity port or bus, such as, for example, parallel ports, serial ports, universal serial busses (USB), or any other similar connectivity port known to those skilled in the art.

Application 216 may be one or more software programs stored in memory 202 and executed by processor 204 of computing device 100. During a planning phase, application 216 guides a clinician through a series of steps to identify a target, size the target, size a treatment zone, and/or determine an access route to the target for later use during the procedure phase. In some embodiments, application 216 is loaded on computing devices in an operating room or other facility where surgical procedures are performed, and is used as a plan or map to guide a clinician performing a surgical procedure, but without any feedback from ablation device 130 used in the procedure to indicate where ablation device 130 is located in relation to the plan. In other embodiments, system 10 provides computing device 100 with data regarding the location of ablation device 130 within the body of the patient, such as by EM tracking, which application 216 may then use to indicate on the plan where ablation device 130 is located.

Application 216 may be installed directly on computing device 100, or may be installed on another computer, for example, a central server, and opened on computing device 100 via network interface 208. Application 216 may run natively on computing device 100, as a web-based application, or any other format known to those skilled in the art. In some embodiments, application 216 will be a single software program having all of the features and functionality described in this disclosure. In other embodiments, application 216 may be two or more distinct software programs providing various parts of these features and functionality. For example, application 216 may include one software program for use during the planning phase, and a second software program for use during the procedure phase of the microwave ablation treatment. In such instances, the various software programs forming part of application 216 may be enabled to communicate with each other and/or import and export various settings and parameters relating to the microwave ablation treatment and/or the patient to share information. For example, a treatment plan and any of its components generated by one software program during the planning phase may be stored and exported to be used by a second software program during the procedure phase.

Application 216 communicates with a user interface 218 that generates a user interface for presenting visual interactive features to a clinician, for example, on display 206 and for receiving clinician input, for example, via a user input device. For example, user interface 218 may generate a graphical user interface (GUI) and output the GUI to display 206 for viewing by a clinician.

Computing device 100 is linked to display 120, thus enabling computing device 100 to control the output on display 120 along with the output on display 206. Computing device 100 may control display 120 to display output which is the same as or similar to the output displayed on display 206. For example, the output on display 206 may be mirrored on display 120. Alternatively, computing device 100 may control display 120 to display different output from that displayed on display 206. For example, display 120 may be controlled to display guidance images and information during the microwave ablation procedure, while display 206 is controlled to display other output, such as configuration or status information.

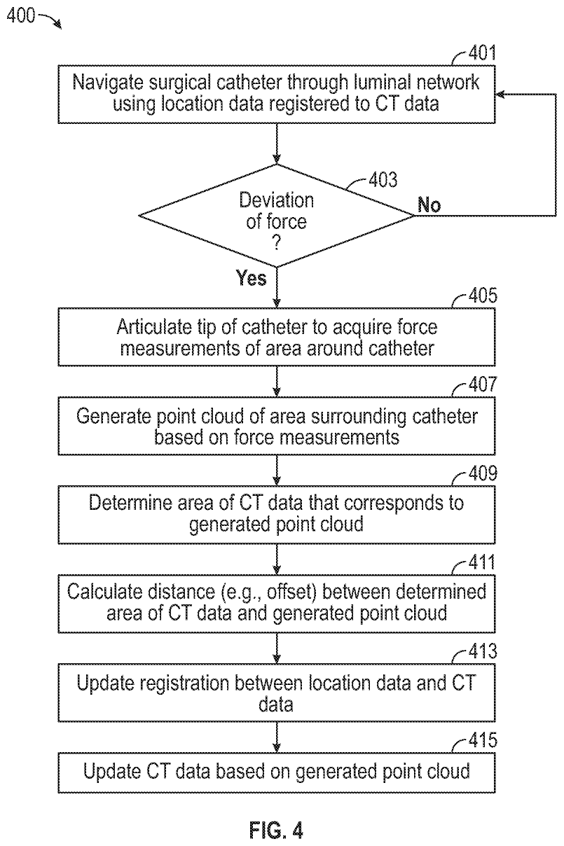

Turning to FIG. 4, a method for dynamically updating registration between location data and image data during a surgical navigation procedure based on force measurements acquired by a force sensor located at a distal tip of a surgical navigation catheter is illustrated and described as method 400. Method 400 is described as being executed by computing device 100, but some or all of the steps of method 400 may be implemented by one or more other components of the system 10, alone or in combination. Additionally, although method 400 is illustrated and described as including specific steps, and is described as being carried out in a particular order, it is understood that method 400 may include some or all of the steps described and may be carried out in any order not specifically described.

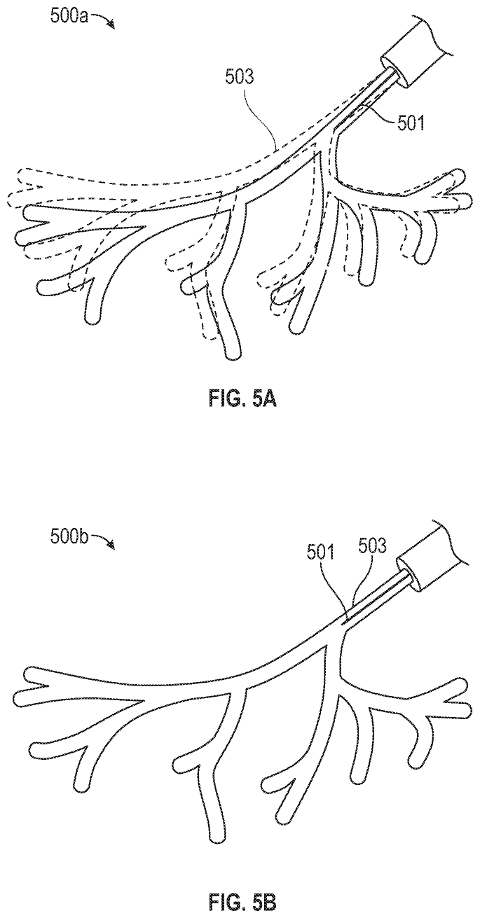

Method 400 begins at step 401 where a catheter 12 is navigated manually or robotically through a patient's luminal network along a planned path using location data of the catheter 12 that is registered to image data of the patient's luminal network. In particular, as described above computing device 100 registers location data of the catheter 12 acquired by tracking system 50 to image data of the luminal network. During navigation, a graphical user interface may be displayed including a image data rendering 503 (FIG. 5A), derived from the image data, and a catheter rendering 501 (FIG. 5A), derived from the location data of the location sensor 44 of the catheter 12 as tracked by the tracking system 50, relative to the image data rendering 503 based on the registration between the location data and the image data. In FIG. 5A, a graphical user interface 500a is shown illustrating the misalignment of the location data and the image data resulting from image to body divergence (e.g., CT to body divergence) as the catheter rendering 501 is displayed outside of an airway lumen of the image data rendering 503. In FIG. 5B, a graphical user interface 500b is shown illustrating the alignment of the location data and the image data subsequent to the execution of method 400.

As described above, the catheter 12 may be navigated robotically with the computing device 100 or another component of system 10 controlling automatic steering and navigation. The automatic steering and navigation may utilize the fore sensor 126 data to maintain the catheter 12 in the center of the lumen through which it is traversing, thereby reducing the risk of damaging the walls of the luminal network during navigation. For example, the computing device 100 may implement a force gradient descent algorithm to follow the least resistive path within the lumen, to maintain the lowest force measurements, thus, ensuring that the catheter 12 remains within the center of the lumen.

During navigation of the catheter 12 along the planned path, in step 403, the computing device 100 determines if the force measured by the force sensor 126 of the catheter 12 deviates from a force expected to be imparted upon the catheter 12 corresponding to the location of the catheter from the location data. For example, expected force values are known for every location of the catheter 12 along a planned pathway. Once the actual force value, as measured by the force sensor 126 at a given location of the catheter 12, deviates (e.g., exceeds a preconfigured threshold) from the expected force value for the given location of the catheter 12, then the computing device 100 may determine that the location data is not aligned with the image data, and thus, the determined location of the catheter 12 relative to the image data is inaccurate. For example, in step 403, either: 1) a force may be sensed by the force sensor 126 when a graphical user interface displays the catheter rendering 501 within a center of a lumen of the image data rendering 503; or 2) the force sensor 126 does not sense any force against the catheter 12 when a graphical user interface displays the catheter rendering 501 abutting a wall of a lumen within the image data rendering 503. In either case, computing device 100 determines that a deviation is detected in step 403 (YES in 403).

When no deviation is detected in step 403 (NO in step 403), then method 400 does not perform any correction. However, when a deviation is detected in step 403 (YES in 403), then method 400 proceeds to step 405. In step 405, the tip of the catheter 12 is articulated to acquire force measurements of the area around the catheter 12 using force sensor 126. The articulation of the tip of the catheter 12 may be carried out automatically via a robotically controlled catheter 12. In aspects, computing device 100 may notify a clinician that a deviation is detected and may instruct the clinician to manually articulate the tip of the catheter 12 to acquire force measurements of the area around the catheter 12 using force sensor 126. In aspects, the articulation of the tip of the catheter 12 may be carried out while the catheter 12 is fixed in one position along the pathway or the articulation of the catheter 12 may be carried out while the catheter 12 is being advanced and/or retracted along the pathway to generate a larger (e.g., elongated) point cloud.

In step 407, the computing device 100 generates a point cloud of the area surrounding the catheter 12 based on the force measurements acquired by the force sensor 126 in step 405. In step 409, the computing device 100 determines which area of the image data corresponds to the generated point cloud, which may be assumed to be proximate the tracked location of the catheter 12. In addition to assuming that the area of the image data that corresponds to the generated point cloud is proximate the tracked location of the catheter, the computing device 100 may determine the corresponding area by using shape and surface-texture matching techniques. Once a corresponding area is detected in step 409, the computing device 100 calculates the offset between the point cloud and the corresponding location in the image data in step 411. For example, in step 411, the computing device 100 may calculate the distance between a center or an edge of the determined corresponding area in the image data and a center or an edge of the generated point cloud.

In step 413, the computing device 100 updates the registration between the location data and the image data based on the offset calculated in step 411. Graphical user interface 500b (FIG. 5B) illustrates the catheter rendering 501 displayed relative to the image data rendering 503 after the registration update is executed in step 413, specifically, with the catheter rendering 501 aligned with the center of a lumen of the image data rendering 503. In an aspect, one of the image data or the location data is shifted toward the other the value of the calculated distance between a center or an edge of the determined corresponding area in the image data and a center or an edge of the generated point cloud. In another aspect, each of the image data and the location data are shifted toward each other a value equal to half of the calculated distance between a center or an edge of the determined corresponding area in the image data and a center or an edge of the generated point cloud.

In aspects, method 400 may additionally include step 415 where the three-dimensional data of the point cloud generated in step 407 is used to update the image data. For example, incomplete image data, or portions of a rendering generated from the image data that may be of low quality or low confidence may be patched with data derived from the generated point cloud. Updating the image data in step 407 ensures that a rendering generated from the image data which is displayed to a clinician includes a more accurate and up-to-date depiction of the patient's lungs, as opposed to a depiction which is derived from image data that was acquired prior to the navigation procedure.

In addition to utilizing the data derived from the force sensor 126, the computing device 100 may additionally perform tissue-type recognition (e.g., malignant vs. benign) of tissue adjacent the catheter 12 based measurements associated with the tissue's mechanical properties. The mechanical properties of the tissue may be measured using the force sensor 126 of the catheter or via another means. For example, at a biopsy stage, the distal tip of the catheter 12 may be moved, either manually or robotically, to create a distal tip motion for a preconfigured distance (e.g., move left 1 mm) and the computing device 100 may monitor the torque and/or current generated to accomplish the distal tip motion. The computing device 100 then compares the monitored torque and/or current generated to an expected value that would be required if the catheter 12 was interacting with healthy (e.g., benign) tissue. If the difference between the monitored value and the expected value exceeds a preconfigured threshold, then the computing device 100 determines that the catheter 12 is located proximate cancerous (e.g., malignant) tissue. Additionally, in aspects, a dual-channel catheter may include two working channels, enabling one instrument to be inserted through each of the working channels. In such a configuration, the distal end of the dual-channel catheter may be navigated to a branch within the luminal network having two paths extending from the branch and each of the instruments may be navigated through a respective branch to position a target between the two instruments. The electrical impedance between the two instruments may be measured and the computing device 100 may utilize the electrical impedance measurements to determine whether the target is malignant or benign.

Although embodiments have been described in detail with reference to the accompanying drawings for the purpose of illustration and description, it is to be understood that the inventive processes and apparatus are not to be construed as limited thereby. It will be apparent to those of ordinary skill in the art that various modifications to the foregoing embodiments may be made without departing from the scope of the disclosure.

1. A surgical navigation system comprising:
a surgical navigation catheter including a force sensor located at a distal portion of the surgical navigation catheter;
a computing device including a processor and memory storing instructions which, when executed by the processor, cause the computing device to:
register location data, corresponding to locations of the surgical navigation catheter navigating along a path through a luminal network, to image data of the luminal network;
receive force measurements from the force sensor of the surgical navigation catheter;
compare force measurements received from the force sensor to expected force measurements along the path and determine whether force measurements received from the force sensor deviate from expected force measurements along the path; and
update registration of the location data to the image data of the luminal network if it is determined that a force measurement received from the force sensor deviates from an expected force measurement along the path.

2. The surgical navigation system of claim 1, wherein the computing device is configured to:
acquire force measurements of an area around a distal portion of the surgical navigation catheter if it is determined that a force measurement received from the force sensor deviates from an expected force measurement along the path; and
generate a point cloud of the area around the distal portion of the surgical navigation catheter based on the acquired force measurements.

3. The surgical navigation system of claim 2, wherein the computing device is configured to:
determine an area of the image data that corresponds to the generated point cloud; and
update registration of the location data to the image data of the luminal network based on the determined area.

4. The surgical navigation system of claim 2, wherein the computing device is configured to:
determine an area of the image data that corresponds to the generated point cloud;
calculate an offset between the determined area and the generated point cloud; and
update registration of the location data to the image data of the luminal network based on the calculated offset.

5. The surgical navigation system of claim 2, wherein the computing device is configured to update at least a portion of the image data based on the generated point cloud.

6. The surgical navigation system of claim 2, wherein the computing device is configured to:
determine an area of the image data that corresponds to the generated point cloud;
calculate a distance between an edge of the area of the image data and an edge of the generated point cloud; and
update registration of the location data to the image data of the luminal network based on the calculated distance.

7. The surgical navigation system of claim 1, further comprising a display operably coupled to the computing device, wherein the computing device is configured to cause the display to display:
a rendering of the luminal network based on the image data; and
a rendering of the surgical navigation catheter positioned relative to the rendering of the luminal network based on the updated registration of the location data to the image data.

8. The surgical navigation system of claim 1, wherein the computing device is configured to generate a notification if it is determined that a force measurement received from the force sensor deviates from an expected force measurement along the path.

9. The surgical navigation system of claim 1, wherein the computing device is configured to initiate movement of the surgical navigation catheter if it is determined that a force measurement received from the force sensor deviates from an expected force measurement along the path.

10. A surgical navigation system comprising:
a surgical navigation catheter configured to navigate along a path through a luminal network, the surgical navigation catheter including:
a location sensor; and
a force sensor;
a tracking system operably coupled to the surgical navigation catheter and configured to generate location data corresponding to locations of the surgical navigation catheter within the luminal network based on signals received from the location sensor as the surgical navigation catheter is navigated through the luminal network; and
a computing device operably coupled to the surgical navigation catheter and the tracking system, the computing device including a processor and memory storing instructions which, when executed by the processor, cause the computing device to:
register the generated location data to image data of the luminal network;
compare force measurements received from the force sensor to expected force measurements along the path and determine whether force measurements received from the force sensor deviate from expected force measurements along the path; and
update registration of the generated location data to the image data of the luminal network if it is determined that a force measurement received from the force sensor deviates from an expected force measurement along the path.

11. The surgical navigation system of claim 10, wherein the location sensor and the force sensor are disposed at a distal portion of the surgical navigation catheter.

12. The surgical navigation system of claim 10, wherein the computing device is configured to:
acquire force measurements of an area around a distal portion of the surgical navigation catheter if it is determined that a force measurement received from the force sensor deviates from an expected force measurement along the path; and
generate a point cloud of the area around the distal portion of the surgical navigation catheter based on the acquired force measurements.

13. The surgical navigation system of claim 12, wherein the computing device is configured to:
determine an area of the image data that corresponds to the generated point cloud; and
update registration of the generated location data to the image data of the luminal network based on the determined area.

14. The surgical navigation system of claim 12, wherein the computing device is configured to:
determine an area of the image data that corresponds to the generated point cloud;
calculate an offset between the determined area and the generated point cloud; and
update registration of the generated location data to the image data of the luminal network based on the calculated offset.

15. The surgical navigation system of claim 12, wherein the computing device is configured to update at least a portion of the image data based on the generated point cloud.

16. The surgical navigation system of claim 10, further comprising a display operably coupled to the computing device, wherein the computing device is configured to cause the display to display:
a rendering of the luminal network based on the image data; and
a rendering of the surgical navigation catheter positioned relative to the rendering of the luminal network based on the updated registration of the generated location data to the image data.

17. The surgical navigation system of claim 10, wherein the computing device is configured to generate a notification if it is determined that a force measurement received from the force sensor deviates from an expected force measurement along the path.

18. The surgical navigation system of claim 10, wherein the computing device is configured to initiate movement of the surgical navigation catheter if it is determined that a force measurement received from the force sensor deviates from an expected force measurement along the path.

19. A method for enhanced navigation of a surgical navigation catheter along a path of a luminal network, the method including:
comparing force measurements received from a force sensor of the surgical navigation catheter to expected force measurements along the path of the luminal network and determining whether force measurements received from the force sensor deviate from expected force measurements along the path;
acquiring force measurements of an area around a distal portion of the surgical navigation catheter if it is determined that a force measurement received from the force sensor deviates from an expected force measurement along the path;
generating a point cloud of the area around the distal portion of the surgical navigation catheter based on the acquired force measurements; and
updating registration of location data, corresponding to locations of the surgical navigation catheter navigating along the path, to image data of the luminal network based on the generated point cloud.

20. The method of claim 19, further comprising:
displaying a rendering of the luminal network based on the image data; and
displaying a rendering of the surgical navigation catheter positioned relative to the displayed rendering of the luminal network based on the updated registration of the location data to the image data.
